(19) United States
(12) Patent Application Publication
(10) Pub. No.: US 2010/0121212 A1
(43) Pub. Date: May 13, 2010

(54) ADAPTER, APPARATUS AND METHOD FOR EXHALED BREATH MEASUREMENTS

(76) Inventors: Mats Carlsson, Taby (SE); Hans Peter Starck-Johnsson, Stockholm (SE)

Correspondence Address: MORRISON & FOERSTER LLP, 425 MARKET STREET, SAN FRANCISCO, CA, 94105-2482, US

(21) Appl. No.: 12/594,573

(22) PCT Filed: Apr. 4, 2008
(86) PCT No.: PCT/SE08/50395

§ 371 Date: Oct. 2, 2009

(30) Foreign Application Priority Data
Apr. 5, 2007 (SE) 0700869-1

(51) Int. Cl. A61B 5/087

(52) U.S. Cl. 600/538

(57) ABSTRACT

An adapter (1) for an exhaled breath measurement apparatus (2), comprising a first inlet (3) for tidal breathing, a second inlet (4) for air free from one or more component to be measured, through which and a first one-way valve (5) air flows when a test person inhales through the adapter (1) at the first inlet (3), and an outlet (6) for exhaled breath having a second one-way valve (7). A sample tubing (11) is positioned next to the first inlet (3) for tidal breathing for a continuous sample from the exhaled breath immediately inside the adapter (1), upstream the second one-way valve (7) at the outlet (6) for exhaled breath. An exhaled breath measurement apparatus (2) with such adapter (1). A method for measuring exhaled breath during tidal breathing by sampling continuously in an adapter (1) at a first inlet (3) into which a test person breathes, the sampling taking place upstream of an outlet (6) for exhaled breath.

The present invention concerns an adapter for use in an exhaled breath measurement apparatus. Especially an adapter comprising a first inlet for tidal breathing into which a person to be tested tidal breathes, a second inlet for air free from one or more component to be measured, through which second inlet and further through a first one-way valve air flows when the test person inhales through the adapter at the first inlet, and an outlet for exhaled breath provided with a second one-way valve.

The invention also concerns an exhaled breath measurement apparatus provided with such an adapter and a method for measuring at least one component of exhaled breath during tidal breathing.

Usually measurement of one or more components of exhaled breath is performed on one exhalation, the sample preferably taken when alveolar air is exhaled. It can be a problem to receive a sufficient exhalation breath from small children, elderly and people with impaired lung function. Often the flow rate or pressure is insufficient. In these cases it is preferred to take the sample during tidal breathing.

When measuring at least one component of exhaled air when the person to be tested tidal breathes a problem arises with dead space air moving back and forth within the measurement apparatus or a connected adapter. One aim of this invention is to minimize the contamination of the sample with dead space air and thus increase the accuracy of tidal breathing measurements.

According to a first aspect of the invention, one solution to the problem is presented in claim 1 where the adapter according to the preamble is provided with a free end of a sample tubing positioned next to the first inlet for exhaled breath for taking of a continuous sample from the exhaled breath immediately inside the adapter, upstream the second one-way valve provided at the outlet for exhaled breath.

By placing the sampling tubing as close as possible to the inlet for exhaled breath inside the adapter it is ensured that only the dead space in the airways and a possible patient filter will be present in the sample during the exhalation. When the test person inhales it is only the small volume inside the adapter that will be drawn back into the lungs but it will be diluted with all the clean air inhaled through the second inlet.

The rest of the exhaled air flows through the second one-way valve and out of the outlet for exhaled air. Usually, when components of exhaled air are to be measured the sample is taken downstream the one-way valve. This would increase the dead space volume and contaminate the sample both in the beginning and in the end of the exhalation phase.

By taking a continuous sample during tidal breathing the amount of the component to be measured can be continuously measured and followed in a time scale diagram from which a very accurate result can be drawn. In known art a sample is collected for a subsequent measurement of the content of the component to be measured. Collecting the sample will mix all the different phases of the tidal breathing and give a mean result only.

Preferably the sample tubing is connected to the exhaled breath measurement apparatus leading the sample separately from the rest of the exhaled air, which also preferably is lead to the exhaled breath measurement apparatus. In a preferred embodiment the flow of at least the exhaled breath is measured.

For example the flow measurement may be performed during overpressure but be stand-by during sub pressure, i.e. inhalation, or continuously during the whole tidal breathing. It is not required to measure the inhalation flow since it will be more or less the same as the exhalation, at least the volume is the same. Preferably the sample tubing is provided inside a tubing for the rest of the exhaled breath.

According to a second aspect of the present invention an exhaled breath measurement apparatus with the above mentioned adapter is provided. In a preferred embodiment a flow meter for measuring exhaled breath flow is provided in the apparatus.

According to a third aspect of the present invention a method for measuring at least one component of exhaled breath during tidal breathing is provided.

### SHORT DESCRIPTION OF THE DRAWINGS

The present invention will now be described in more detail by means of preferred embodiments referring to the enclosed drawings, in which:

FIG. 1 shows a preferred embodiment of the adapter of the invention in a cross section view,

FIG. 2 shows two diagrams, the above Fractional Exhaled NO FE_{NO}/Time and the below Flow/Time, and

FIG. 3 shows a preferred embodiment of an exhaled breath measurement apparatus according to the present invention.

### DETAILED DESCRIPTION OF PREFERRED EMBODIMENTS

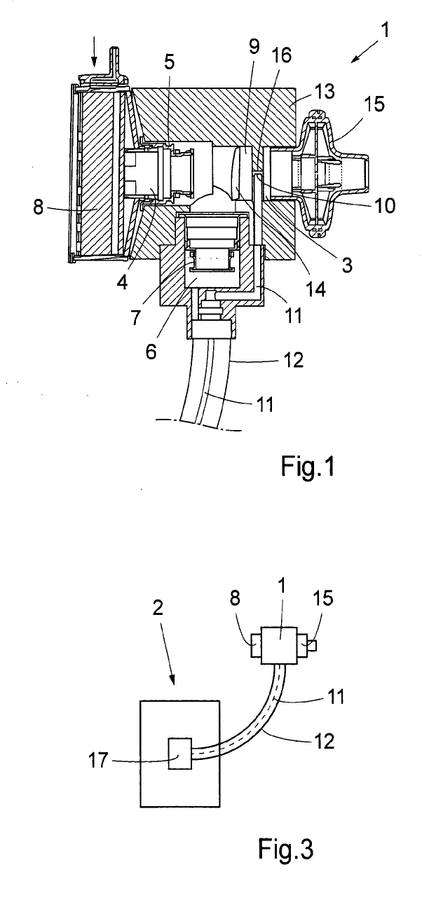

A preferred embodiment of the adapter of the present invention is shown in a cross section view in FIG. 1. An adapter 1 for use in an exhaled breath measurement apparatus 2, se FIG. 3, has a body 13 and is provided with a first inlet 3 for tidal breathing into which inlet 3 a person to be tested tidal breathes. A second inlet 4 for clean air, i.e. air free from the at least one component to be measured, is provided in order to supply clean air to the test person when he or she tidal breathes.

A first one-way valve 5 is arranged in the second inlet 4 so that clean air may flow into the adapter 1 but the first one-way valve 5 will not let out any flow from the adapter 1. An outlet 6 for exhaled breath is provided and in this outlet a second one-way valve 7 is arranged so that the exhaled breath may flow out from the adapter 1 but the second one-way valve 7 will not let any flow into the adapter. In this way it is secured that the test person only inhales clean air through the second inlet 4 via the adapter 1 out through the first inlet 3 and into the test person.

Clean air may be provided from a clean air source or preferably by arranging a filter 8 for filtering out the at least one component to be measured in the second inlet 4, outside the first one-way valve 5. The adapter 1 is preferably small but a very important feature is that the inner void 9 of the adapter 1 is as small as possible in order to minimize the amount of air that could flow back and forth during the tidal breathing. Thus the first and second one-way valves 5 and 7 ought to be arranged adjacent each other and the first inlet 3, for example so the flow paths form a T- or Y-junction.

A free end 10 of a sample tubing 11 is positioned next to the first inlet 3 immediately inside the adapter 1. The sample tubing 11 is provided for taking a continuous sample of the exhaled breath. The sample taking, i.e. the position of the free end 10 of the sample tubing 11, takes place in the exhaled breath flow just inside the adapter 1 upstream, i.e. before in the exhaled breath flow path, the second one-way valve 7 for exhaled breath, which is provided in the outlet 6 for exhaled breath.

The rest of the exhaled breath leaving the adapter 1 through the outlet 6 is preferably led to the exhaled breath measurement apparatus 2 by means of a tubing 12. The sample and the rest of the exhaled breath is lead separately to the apparatus 2. Preferably the sample tubing 11 is provided inside the tubing 12 for the rest of the exhaled breath.

Preferably at least the flow of the exhaled breath is measured, for example by means of a flow meter 14. It can be provided in the adapter 1 or as in the preferred embodiment in the apparatus 2.

For example the flow measurement may be performed during overpressure but be stand-by during sub pressure, i.e. inhalation, or continuously during the whole tidal breathing. It is not required to measure the inhalation flow since it will be more or less the same as the exhalation. This is depending on the lung capacity, which will not particularly change during a test.

In order not to contaminate the adapter 1 with various viruses, bacteria, moist and so on from different test persons a patient filter 15 for filtering out unwanted components, for example the above mentioned, is provided in the first inlet 3. The patient filter 15 could for example be a filter of HEPA type. Preferably a new patient filter 15 is positioned in the first inlet 3 before each test.

Preferably the first inlet 3 has a reduced diameter in a flow path 16 commencing directly at the inlet 3 and ending directly after the free end 10 of the sample tubing 11.

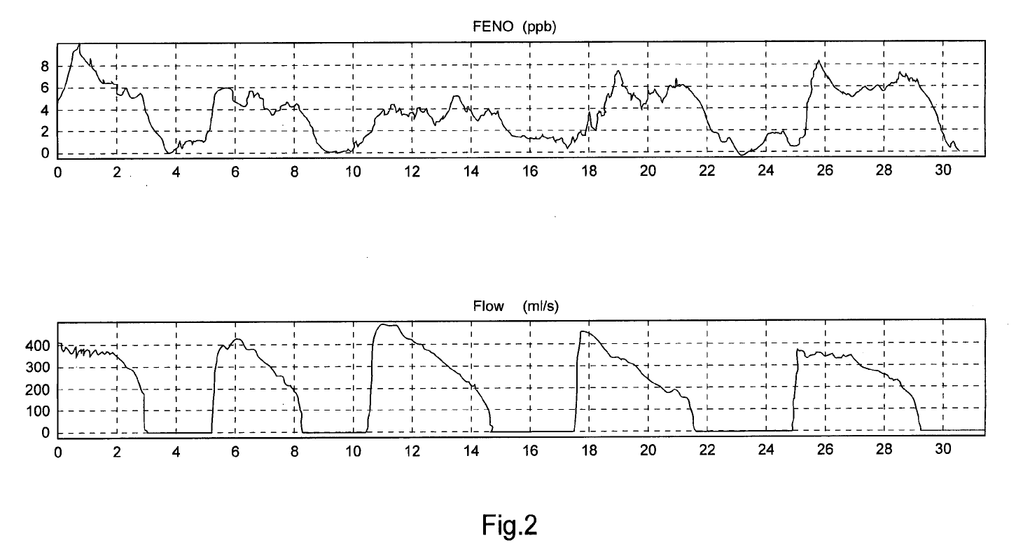

The sample flow in the sample tubing 11 is preferably in the interval 1-15 ml/s and most preferred about 5 ml/s. In FIG. 2 two diagrams are shown. The above one shows the amount of FE_{NO}) (Fractional Exhaled NO) vs Time and the one below shows Flow vs Time, the flow is the rest of the exhaled breath provided to the apparatus in the tubing 12. As can be seen only exhaled flow has been measured.

Thus, in this example the measured component is nitrogen monoxide, NO, but of course any other component of exhaled breath could be measured. During inhalation the diagram shows zero. The rest of the exhaled breath flow can for example be up to 500 ml/s or more.

When the test person exhales a sample will continuously pass through the sampling tube 11 and into a reaction chamber 17. Since the sample tubing 11 has a small inner diameter the flow will be small and thus there will be a lag time until the sample reaches the reaction chamber 17 compared to the metered flow of the rest of the exhaled breath, which is almost momentarily. A lag time compensation is introduced in the soft ware.

The lag time depends on the sample flow and the size and length of the sample tubing 11. It is preferred that the lag time is not too big since it could make the FE_{NO} result to add and mix with the next sample of exhaled breath. Also the fault in the synchronization of the flow measurement may be increased, especially if the lag time differs and is difficult to control.

A slow answer form the reaction chamber 17 may also cause the FE_{NO} result to add and mix with the next sample of exhaled breath. The best results will be achieved if FE_{NO} measurement will have time to reach zero before the next exhalation breath sample is reaching the reaction chamber 17.

In the preferred embodiment the sampling of the exhaled breath is continuous and taken directly at the first inlet 3 for exhalation breath at the same time as the test person tidal breathes. The sample is lead to an apparatus for a continuous measurement, thus no collection of the sample is needed nor wanted since it will mix the different phases of one exhalation breath, and if more than one exhalation breath sample is collected also the different exhalation breaths will be mixed only giving a mean result.

A certain resistance in the adapter 1 is necessary and will also slightly slow down and increase the amplitude of the tidal breathing which could be positive, and for some patients necessary, for reaching zero in between the subsequent exhaled breath samples and would also lead to a smoother tidal breathing flow with less fluctuations in exhaled flow. Preferably the resistance is approximately the same for exhalation and inhalation for a more comfortable respiration.

Preferably a minimum flow resistance is equivalent to a pressure drop of 0.5 mBar at 100 ml/s and preferably a maximum flow resistance is equivalent to a pressure drop of 4 mBar at 100 ml/s. Above this maximum limit tidal breathing is no longer normal and gives an uncomfortable feeling giving extended breaths with a low flow so that the patient feels a lack of air.

It is also conceivable to adapt the flow resistance using a variable flow resistance in the tubing to cause a desired respiration pattern for a specific patient, since with a certain flow resistance the exhale flow will adapt to a corresponding flow interval.

The invention is suitable for measuring components of gases such as for example ethanol, acetone, carbon dioxide, carbon monoxide, oxygen, hydrogen, or nitrogen dioxide.

1. An adapter (1) for use in an exhaled breath measurement apparatus (2), the adapter (1) comprising a first inlet (3) for tidal breathing into which a person to be tested tidal breathes, a second inlet (4) for air free from one or more component to be measured, through which second inlet (4) and further through a first one-way valve (5) air flows when the test person inhales through the adapter (1) at the first inlet (3), and an outlet (6) for exhaled breath provided with a second one-way valve (7), characterised in that a free end (10) of a sample tubing (11) is positioned next to the first inlet (3) for tidal breathing for taking of a continuous sample from the exhaled breath immediately inside the adapter (1), upstream the second one-way valve (7) provided at the outlet (6) for exhaled breath.

2. An adapter (1) in accordance with claim 1, wherein the sample tubing (11) leads into the exhaled breath measurement apparatus (2) leading the sample separately from the rest of the exhaled breath.

3. An adapter according to claim 1, wherein a flow meter (14) is arranged for measurement of at least the flow of the exhaled breath.

4. An adapter (1) according to claim 1, wherein the adapter (1) is connected at the exhaled breath outlet (6) to the exhaled breath measurement apparatus (2) via an exhaled breath tubing (12).

5. An exhaled breath measurement apparatus (2), characterised in that it is provided with an adapter (1) according to claim 1.

6. An apparatus according to claim 5, wherein a flow meter (14) is provided for measuring the exhaled breath flow.

7. An apparatus (2) according to claim 5, wherein the adapter (1) is connected at the exhaled breath outlet (6) to the exhaled breath measurement apparatus (2) via an exhaled breath tubing (12).

8. A method for measuring at least One component of exhaled breath during tidal breathing, the method comprising sampling continuously in the tidal breathing flow flowing through an adapter (1), the sampling taking place directly at a first inlet (3) of the adapter (1) into which first inlet (3) a person to be tested tidal breathes, the sampling taking place upstream of a one-way valve (7) at an outlet (6) for exhaled breath.

9. A method according to claim 8, whereby the continuous sample is lead to the exhaled breath measurement apparatus (2) separately from the rest of the exhaled breath.

10. A method according to claim 8, whereby the flow of the exhaled breath is measured by means of a flow meter (14).

11. An adapter according to claim 2, wherein a flow meter (14) is arranged for measurement of at least the flow of the exhaled breath.

12. An adapter (1) according to claim 2, wherein the adapter (1) is connected at the exhaled breath outlet (6) to the exhaled breath measurement apparatus (2) via an exhaled breath tubing (12).

13. An adapter (1) according to claim 3, wherein the adapter (1) is connected at the exhaled breath outlet (6) to the exhaled breath measurement apparatus (2) via an exhaled breath tubing (12).

14. An apparatus (2) according to claim 6, wherein the adapter (1) is connected at the exhaled breath outlet (6) to the exhaled breath measurement apparatus (2) via an exhaled breath tubing (12).

15. A method according to claim 9, whereby the flow of the exhaled breath is measured by means of a flow meter (14).
